(19) United States
(12) Patent Application Publication
(10) Pub. No.: US 2010/0063299 A1
(43) Pub. Date: Mar. 11, 2010

(54) Process for Preparing Irbesartan

(76) Inventors: Udhaya Kumar, Pondicherry (IN); Praveen Kumar Neela, Hyderabad (IN); Nitin Sharadchandra Pradhan, Maharashtra (IN); Jon Valgeirsson, Hafnarfjordur (IS)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/530,039

(22) PCT Filed: Mar. 6, 2008
(86) PCT No.: PCT/IB2008/000973

§ 371 Date: Nov. 12, 2009

(30) Foreign Application Priority Data
Mar. 6, 2007 (IN) 439/CHE/2007

(51) Int. Cl. C07D 257/04

(52) U.S. Cl. 548/254

(57) ABSTRACT

Disclosed herein is an improved, commercially viable and industrially advantageous process for the preparation of Irbesartan, or a pharmaceutically acceptable salt thereof, in high yield and purity.

### CROSS REFERENCE TO RELATED APPLICATION

This application claims priority from Indian Provisional Application Ser. No. 439/CHE/2007 filed Mar. 6, 2007, which is hereby incorporated by reference in its entirety.

### FIELD OF THE DISCLOSURE

Disclosed herein is an improved, commercially viable and industrially advantageous process for the preparation of Irbesartan, or a pharmaceutically acceptable salt thereof, in high yield and purity.

### BACKGROUND OF THE INVENTION

U.S. Pat. No. 5,270,317 discloses a variety of N-substituted heterocyclic derivatives and their salts, processes for their preparation, pharmaceutical compositions comprising the derivatives, and method of use thereof. These compounds are angiotensin II receptor antagonists. Among them, Irbesartan, 2-n-butyl-3-[[2′-(tetrazol-5-yl)biphenyl-4-yl]methyl]-1,3-diazaspiro[4.4]non-1-en-4-one, is a potent, long-acting angiotensin II receptor antagonist that is especially useful in the treatment of cardiovascular ailments such as hypertension and heart failure, as well as in preventing disorders of central nervous system, glaucoma, diabetic retinopathy, and diabetic nephropathy. Irbesartan is represented by the following structural formula I:

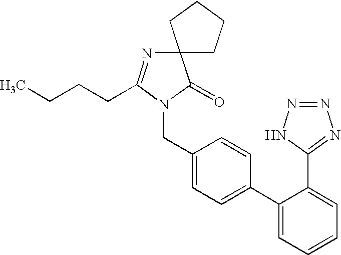

Various processes for the preparation of Irbesartan and related compounds are disclosed in U.S. Pat. Nos. 5,270,317 and 5,629,331, and PCT Publication Nos. WO 2005/051943 A1 and WO 2007/013101 A1.

As per the processes described in the U.S. Pat. No. 5,270,317 (herein after referred to as the '317 patent), Irbesartan is prepared by reaction of 2-n-Butyl-4-spirocylopentane-2-imidazolin-5-one with 4-bromomethyl-2-cyanobiphenyl in the presence of sodium hydroxide, followed by a column chromatography separation to produce 1-[(2′-cyanobiphenyl-4-yl)methyl]-2-n-butyl-4-spirocyclopentane-2-imidazolin-5-one, which by reaction with tributyltin azide and trityl chloride followed by deprotection with HCl to produce Irbesartan.

Irbesartan obtained by the process described in the '317 patent does not have satisfactory purity. Unacceptable amounts of impurities are formed along with Irbesartan. The yield of Irbesartan obtained is very poor and the process involves column chromatographic purifications. Methods involving column chromatographic purifications are generally undesirable for large-scale operations, thereby making the process commercially unfeasible. The process used in the '317 patent also suffers from disadvantages such as high cost of reagents, the use of additional reagents such as tributyltin azide and trityl chloride, low yields of the product, extra purification steps to obtain the final product and health hazards. The use of tributyltin azide is not advisable for scale up operations.

U.S. Pat. No. 5,629,331 (hereinafter referred to as the '331 patent) describes a process for the preparation of Irbesartan wherein 2-n-butyl-3-[[2′-cyanobiphenyl-4-yl]methyl]-1,3-diazaspiro-[4.4]non-1-en-4-one is treated with sodium azide in the presence of triethylamine hydrochloride in an inert polar aprotic solvent such as 1-methylpyrrolidin-2-one at a temperature of 121-123° C. The solvent used is costly and not easily recovered thereby making the process unsuitable for commercial scale production. Isolation of Irbesartan from the reaction mixture is tedious and requires several critical layer separations and layer filtrations. Moreover, Irbesartan obtained by the process described in the '331 patent does not have satisfactory purity. Unacceptable amounts of impurities are formed along with Irbesartan, thus resulting in a poor product yield.

PCT Publication No. WO 2007/013101 (hereinafter referred to as the '101 application) describes a process for the preparation of Irbesartan wherein 2-n-butyl-3-[[2′-cyanobiphenyl-4-yl]methyl]-1,3-diazaspiro-[4.4]non-1-en-4-one is treated with sodium azide in the presence of triethylamine and acetic acid.

Irbesartan obtained by the process described in the '101 application does not have satisfactory purity and the process produces poor product yield.

Based on the aforementioned drawbacks, the prior art processes may be unsuitable for preparation of Irbesartan in commercial scale operations.

A need remains for an improved and commercially viable process of preparing a substantially pure Irbesartan, to resolve the problems associated with the processes described in the prior art, and that will be suitable for large-scale preparation. Desirable process properties include non-hazardous and environmentally friendly reagents, reduced cost, greater simplicity, increased purity, and increased yield of the product.

### SUMMARY OF THE INVENTION

The present inventors have surprisingly found that 2-n-butyl-3-[[2′-(tetrazol-5-yl)biphenyl-4-yl]methyl]-1,3-diazaspiro[4.4]non-1-en-4-one (Irbesartan of formula I) can be prepared in high purity and with high yield by reacting 2-n-butyl-3-[[2′-cyanobiphenyl-4-yl]methyl]-1,3-diazaspiro-[4.4]non-1-en-4-one with an alkalimetal azide and tri(C₁₋₄)alkylamine hydrohalide such as triethylamine hydrochloride in the presence of a phase transfer catalyst in a non polar aprotic solvent.

In one aspect, provided herein is an efficient, convenient, commercially viable and environment friendly process for the preparation of Irbesartan in an 84-90% overall yield. Advantageously, the reagents used for present invention are easy to handle at commercial scale and are also less expensive reagents and less hazardous than in many prior art processes.

In another aspect, the present invention provides substantially pure Irbesartan or a pharmaceutically acceptable salt thereof having relatively low content of one or more organic volatile impurities.

### DETAILED DESCRIPTION OF THE INVENTION

In accordance with the present invention, there is provided a process for the preparation of 2-n-butyl-3-[[2′-(tetrazol-5-yl)biphenyl-4-yl]methyl]-1,3-diazaspiro[4.4]non-1-en-4-one (Irbesartan) or a pharmaceutically acceptable salt thereof, which comprises:
a) reacting 2-n-butyl-3-[[2′-cyanobiphenyl-4-yl]methyl]-1,3-diazaspiro-[4.4]non-1-en-4-one with an alkali metal azide and tri(C₁₋₄)alkylamine hydrohalide in the presence of a phase transfer catalyst in a non-polar aprotic solvent to produce an alkaline salt of 2-n-butyl-3-[[2′-(tetrazol-5-yl)biphenyl-4-yl]methyl]-1,3-diazaspiro[4.4]non-1-en-4-one; and
b) neutralizing the alkaline salt of 2-n-butyl-3-[[2′-(tetrazol-5-yl)biphenyl-4-yl]methyl]-1,3-diazaspiro[4.4]non-1-en-4-one in aqueous medium with an acid to produce Irbesartan and optionally converting the Irbesartan obtained into its pharmaceutically acceptable salts thereof.

The process can produce substantially pure Irbesartan which can be isolated from the reaction mixture in conventional manner, for example by precipitation and washing.

The tri(C₁₋₄)alkylamine is favorably triethylamine. The hydrohalide is aptly a hydrochloride or hydrobromide of which hydrochloride is presently favored. The presently preferred tri(C₁₋₄)alkylamine hydrohalide is triethylamine hydrochloride.

Exemplary phase transfer catalysts include, but are not limited to, ammonium salts such as tricaprylylmethylammonium chloride (Aliquat® 336), tetra-n-butylammonium bromide (“TBAB”), benzyltriethylammonium chloride (“TEBA”), cetyltrimethylammonium bromide, cetylpyridinium bromide, N-benzylquininium chloride, tetra-n-butylammonium chloride, tetra-n-butylammonium hydroxide, tetra-n-butylammonium iodide, tetra-ethylammonium chloride, benzyltributylammonium bromide, benzyltriethylammonium bromide, hexadecyltriethylammonium chloride, tetramethylammonium chloride, hexadecyltrimethyl ammonium chloride, octyltrimethylammonium chloride, and combinations comprising one or more of the foregoing catalysts. Specific phase transfer catalysts are tricaprylylmethylammonium chloride, tetra-n-butylammonium bromide, benzyltriethylammonium chloride, and combinations comprising one or more of the foregoing catalysts. More specific phase transfer catalyst is tetra-n-butylammonium bromide.

Preferable alkali metal azide used in step-(a) is sodium azide.

Exemplary non polar aprotic solvents used in step-(a) include, but are not limited to, hydrocarbon solvents such as toluene, xylene, cyclohexane, n-octane, and mixtures thereof. Specific non polar aprotic solvents are toluene, xylene, and a mixture thereof.

The reaction in step-(a) is carried out at a temperature of 25° C. to the reflux temperature of the solvent used, specifically at a temperature of 50° C. to the reflux temperature of the solvent used, more specifically at a temperature of 80° C. to the reflux temperature of the solvent used, and most specifically at the reflux temperature of the solvent used.

As used herein, “reflux temperature” means the temperature at which the solvent or solvent system refluxes or boils at atmospheric pressure.

Usually, about 1.5 to 5.0 moles, specifically, about 2.0 to 2.5 moles of alkali metal azide is used per 1 mole of 2-n-butyl-3-[[2′-cyanobiphenyl-4-yl]methyl]-1,3-diazaspiro-[4.4]non-1-en-4-one.

Usually, about 1.5 to 7.0 moles, specifically, about 2.0 to 3.5 moles of triethylamine hydrochloride is used per 1 mole of 2-n-butyl-3-[[2′-cyanobiphenyl-4-yl]methyl]-1,3-diazaspiro-[4.4]non-1-en-4-one.

The reaction mass containing the alkaline salt of 2-n-butyl-3-[[2′-(tetrazol-5-yl)biphenyl-4-yl]methyl]-1,3-diazaspiro[4.4]non-1-en-4-one obtained in step-(a) may be subjected to usual work up such as washings, extractions etc. The reaction mass may be used directly in the next step to produce Irbesartan, or the alkaline salt of 2-n-butyl-3-[[2′-(tetrazol-5-yl)biphenyl-4-yl]methyl]-1,3-diazaspiro[4.4]non-1-en-4-one may be isolated and then used in the next step.

The neutralization reaction in step-(b) is carried out by adjusting the pH of the solution to below about 4.0 and specifically to 2.0-4.0 with an acid.

Preferable acid used in step-(b) is a mineral acid such as sulfuric acid, hydrochloric acid and phosphoric acid. More preferable mineral acid is hydrochloric acid.

Specifically aqueous solution of acid may be used to adjust the pH and more specifically dilute aqueous acid may be used.

In one embodiment, Irbesartan obtained in step-(b) is isolated as solid from a suitable organic solvent by methods usually known in the art such as cooling, partial removal of the solvent from the solution, addition of precipitating solvent or a combination thereof.

The product thus obtained in step-(b) may be further or additionally dried to achieve the desired residual solvent values. For example, the product may be further or additionally dried in a tray drier, or dried under vacuum and/or in a Fluid Bed Drier. If desired, the solution containing Irbesartan can be treated with activated charcoal and filtered while hot or the slurry containing the pure Irbesartan may be cooled prior to filtration.

The phase transfer catalyst in the presence of non-polar aprotic solvent used for the cyclization, allows the product to be easily isolated and purified, thereby producing a product with 84-90% overall yield.

The Irbesartan or a pharmaceutically acceptable salt thereof obtained by the process disclosed herein, have a purity (measured by High Performance Liquid Chromatography, hereinafter referred to as ‘HPLC’) greater than about 99.50%, specifically greater than about 99.90%, and more specifically greater than about 99.95%.

The use of inexpensive, non-hazardous, readily available and easy to handle reagents allows the process disclosed herein to be suitable for preparation of Irbesartan at lab scale and in commercial scale operations.

According to another aspect of the present invention, there is provided substantially pure Irbesartan or a pharmaceutically acceptable salt thereof have a relatively low content of one or more organic volatile impurities and reduced level of tin content.

The Irbesartan obtained by the process disclosed herein, having less than about 50 parts per million (ppm) o-xylene, less than about 200 ppm toluene, less than about 200 ppm N,N-dimethylformamide, less than about 200 ppm ethyl acetate, less than about 200 ppm methyl tert-butyl ether, and less than about 50 ppm triethylamine. Specifically the Irbesartan obtained by the process disclosed herein, having less than about 10 parts per million (ppm) o-xylene, less than about 20 ppm toluene, less than about 20 ppm N,N-dimethylformamide, less than about 20 ppm ethyl acetate, less than about 20 ppm methyl tert-butyl ether, and less than about 10 ppm triethylamine.

Specifically Irbesartan obtained by the process disclosed herein having the overall level of organic volatile impurities less than about 200 ppm, and more specifically less than about 20 ppm. Such Irbesartan will often contain hydrocarbon impurities such as o-xylene or toluene but at less than the stated levels.

It is advantageous that the Irbesartan obtained by the process disclosed herein may have a level of tin content of less than 5 ppm, and more specifically less than 2 ppm. The avoidance of tin catalysts frequently used in the prior art when making Irbesartan is a particular advantage of this invention.

The term “substantially pure Irbesartan or a pharmaceutically acceptable salt thereof”, refers to the Irbesartan or a pharmaceutically acceptable salt thereof having purity greater than about 99.5%, specifically greater than about 99.90%, and more specifically greater than about 99.95% measured by HPLC.

2-n-Butyl-3-[[2′-cyanobiphenyl-4-yl]methyl]-1,3-diazaspiro-[4.4]non-1-en-4-one used as starting material in step-(a) may be obtained by processes described in the prior art, for example by the process described in the U.S. Pat. No. 5,270,317.

Pharmaceutically acceptable salts of Irbesartan can be prepared in high purity by using the substantially pure Irbesartan obtained by the methods disclosed herein, by known methods, for example as described in U.S. Pat. No. 5,270,317.

### EXPERIMENTAL

### The Purity was Measured by High Performance Liquid Chromatography by Using Waters, Alliance 2695 HPLC System Having Dual Wavelength UV Detector Under the Following Conditions

### Column: Sunfire C18 150×4.6 mm, 3.5 μm, Make: Waters,

Column oven temperature: 25° C.

### Detection: UV at 220 nm

Flow rate: 0.5 mL/minute
Injection volume: 20 μl
Run time: 45 min

### Diluent: Methanol

The following examples are given for the purpose of illustrating the present disclosure and should not be considered as limitation on the scope or spirit of the disclosure.

### Reference Examples

### Reference Example 1

### Preparation of 2-n-butyl-3-[[2′-cyanobiphenyl-4-yl]methyl]-1,3-diazaspiro-[4.4]non-1-en-4-one

2-(n-Butyl)-1,3-diazaspiro[4.4]non-1-ene-4-one hydrochloride (8.9 g) and N,N-dimethylformamide (70 ml) were taken into a round bottom flask followed by the addition of sodium hydroxide (3.6 g) at 25-30° C. The reaction mixture was stirred for 10 minutes followed by the addition of 4-(bromomethyl)-2′-cyanobiphenyl (10.0 g) and then the resulting mass was stirred for 6 hours at 25-30° C. The reaction mass was diluted with water (100 ml) followed by extraction with toluene. The toluene was completely distilled out and the product was isolated by using methyl tertiary butyl ether (20 ml) to produce 2-n-butyl-3-[[2′-cyanobiphenyl-4-yl]methyl]-1,3-diazaspiro-[4.4]non-1-en-4-one (Yield: 86%, HPLC purity: 99.2%)

### Reference Example 2

### Preparation of 2-n-butyl-3-[[2′-cyanobiphenyl-4-yl]methyl]-1,3-diazaspiro-[4.4]non-1-en-4-one

2-(n-Butyl)-1,3-diazaspiro[4.4]non-1-ene-4-one (7.5 g) and N,N-dimethylformamide (70 ml) were taken into a round bottom flask followed by the addition of sodium hydroxide (3.6 g) at 25-30° C. The reaction mixture was stirred for 10 minutes followed by the addition of 4-(bromomethyl)-2′-cyanobiphenyl (10.0 g) and then the resulting mass was stirred for 6 hours at 25-30° C. The reaction mixture was diluted with water (100 ml) followed by extraction with toluene. Toluene was completely distilled out and the product was isolated by using methyl tertiary butyl ether (20 ml) to produce 2-n-butyl-3-[[2′-cyanobiphenyl-4-yl]methyl]-1,3-diazaspiro-[4.4]non-1-en-4-one (Yield: 86%, HPLC purity: 99.0).

### Examples

### Example 1

### Preparation of 2-n-butyl-3-[[2-(tetrazol-5-yl)biphenyl-4-yl]methyl]-1,3-diazaspiro[4.4]non-1-en-4-one (Irbesartan)

Triethylamine hydrochloride (12.5 g), sodium azide (3.4 g), tetrabutylammonium bromide (1.0 g) and 2-n-butyl-3-[[2′-cyanobiphenyl-4-yl]methyl]-1,3-diazaspiro-[4.4]non-1-en-4-one (10 g) were taken in o-xylene and heated at 125-130° C. under stirring for 24 hours. The reaction mixture was cooled at 25-30° C. This was followed by the addition of water (30 ml) and 30% sodium hydroxide solution (10 ml) under 30 minutes. The aqueous layer was separated from the settled reaction mixture and washed with xylene (20 ml) followed by the addition of sodium nitrite (3.0 g). Ethyl acetate (70 ml) was added to the aqueous phase and adjusted the pH to about 2.0-4.0 with 6N HCl. The precipitated solid was stirred for 1 hour, filtered washed with water (40 ml) and ethyl acetate (40 ml) to produce Irbesartan (Yield: 85%, HPLC Purity: 99.90%);

Level of organic volatile impurities: o-xylene—3 parts per million (ppm), toluene—5 ppm, N,N-dimethylformamide—Not detected, ethyl acetate—Not detected, methyl tert-butyl ether—Not detected, triethylamine—Not detected, tin—Not detected

### Example 2

### Preparation of 2-n-butyl-3-[[2′-(tetrazol-5-yl)biphenyl-4-yl]methyl]-1,3-diazaspiro[4.4]non-1-en-4-one (Irbesartan)

Triethylamine hydrochloride (45 g), sodium azide (13 g), tetrabutylammonium bromide (2.5 g) and 2-n-butyl-3-[[T-cyanobiphenyl-4-yl]methyl]-1,3-diazaspiro-[4.4]non-1-en-4-one (50 g) were taken in o-xylene and heated at 125-130° C. under stirring for 40 hours. The reaction mixture was cooled at 25-30° C. This was followed by the addition of water (150 ml) and 30% sodium hydroxide solution (50 ml) under 30 minutes. The aqueous layer was separated from the settled reaction mixture and washed with xylene (100 ml). Ethyl acetate (350 ml) was added to aqueous phase and adjusted the pH at about 2.0-4.5 with 6N HCl. The precipitated solid was stirred for 1 hour and filtered washed with water (200 ml) and ethyl acetate (200 ml) to produce Irbesartan (Yield: 85%, HPLC Purity: 99.95%).

Level of organic volatile impurities: o-xylene—2 parts per million (ppm), toluene—6 ppm, N,N-dimethylformamide—Not detected, ethyl acetate—Not detected, methyl tert-butyl ether—Not detected, triethylamine—Not detected and tin—Not detected.

1. A process for the preparation of 2-n-butyl-3-[[2′-(tetrazol-5-yl)biphenyl-4-yl]methyl]-1,3-diazaspiro[4.4]non-1-en-4-one (Irbesartan) or a pharmaceutically acceptable salt thereof, which comprises:
a) reacting 2-n-butyl-3-[[2′-cyanobiphenyl-4-yl]methyl]-1,3-diazaspiro-[4.4]non-1-en-4-one with an alkali metal azide and tri(C₁₋₄)alkylamine hydrohalide in the presence of a phase transfer catalyst in a non-polar aprotic solvent to produce an alkaline salt of 2-n-butyl-3-[[2′-(tetrazol-5-yl)biphenyl-4-yl]methyl]-1,3-diazaspiro[4.4]non-1-en-4-one; and
b) neutralizing the alkaline salt of 2-n-butyl-3-[[2′-(tetrazol-5-yl)biphenyl-4-yl]methyl]-1,3-diazaspiro[4.4]non-1-en-4-one in aqueous medium with an acid to produce Irbesartan and optionally converting the Irbesartan obtained into its pharmaceutically acceptable salts thereof.

2. The process of claim 1, wherein the tri(C₁₋₄)alkyl hydrohalide is triethylamine hydrochloride.

3. The process of claim 1 or claim 2, wherein the phase transfer catalyst is selected from the group consisting of ammonium salts such as tricaprylylmethylammonium chloride (Aliquat® 336), tetra-n-butylammonium bromide (“TBAB”), benzyltriethylammonium chloride (“TEBA”), cetyltrimethylammonium bromide, cetylpyridinium bromide, N-benzylquininium chloride, tetra-n-butylammonium chloride, tetra-n-butylammonium hydroxide, tetra-n-butylammonium iodide, tetra-ethylammonium chloride, benzyltributylammonium bromide, benzyltriethylammonium bromide, hexadecyltriethylammonium chloride, tetramethylammonium chloride, hexadecyltrimethyl ammonium chloride, octyltrimethylammonium chloride, and combinations comprising one or more of the foregoing catalysts.

4. The process of claim 3, wherein the phase transfer catalyst is selected from the group consisting of tricaprylylmethylammonium chloride, tetra-n-butylammonium bromide, benzyltriethylammonium chloride, and combinations comprising one or more of the foregoing catalysts.

5. The process of claim 4, wherein the phase transfer catalyst is tetra-n-butylammonium bromide.

6. The process of claim 1, wherein the alkali metal azide used in step-(a) is sodium azide.

7. The process of claim 1, wherein the non polar aprotic solvent used in step-(a) is selected from the group consisting of toluene, xylene, cyclohexane, n-octane, and mixtures thereof.

8. The process of claim 7, wherein the non polar aprotic solvent is toluene, xylene or a mixture thereof.

9. The process of claim 1, wherein the alkali metal azide is used in a molar ratio of 1.5 to 5.0 moles per 1 mole of 2-n-butyl-3-[[2′-cyanobiphenyl-4-yl]methyl]-1,3-diazaspiro-[4.4]non-1-en-4-one.

10. The process of claim 1, wherein the triethylamine hydrochloride is used in a molar ratio of 1.5 to 7.0 moles per 1 mole of 2-n-butyl-3-[[2′-cyanobiphenyl-4-yl]methyl]-1,3-diazaspiro-[4.4]non-1-en-4-one.

11. The process of claim 1, wherein the neutralization reaction is carried out by adjusting the pH of the solution to below about 4.0 with an acid.

12. The process of claim 11, wherein the pH of the solution is adjusted to 2.0-4.0.

13. The process of claim 1, wherein the acid used in step-(b) is a mineral acid selected from the group consisting of sulfuric acid, hydrochloric acid and phosphoric acid.

14. The process of claim 13, wherein the mineral acid is hydrochloric acid.

15. The process of claim 1, wherein the Irbesartan or a pharmaceutically acceptable salt thereof obtained has a purity of greater than about 99.50% as measured by HPLC.

16. The process of claim 15, wherein the Irbesartan or a pharmaceutically acceptable salt has a purity of greater than about 99.90% as measured by HPLC.

17. The process of claim 16, wherein the Irbesartan or a pharmaceutically acceptable salt has a purity of greater than about 99.95% as measured by HPLC.

18. A substantially pure Irbesartan having less than about 50 parts per million (ppm) o-xylene, less than about 200 ppm toluene, less than about 200 ppm N,N-dimethylformamide, less than about 200 ppm ethyl acetate, less than about 200 ppm methyl tert-butyl ether, and less than about 50 ppm triethylamine.

19. The compound of claim 18, wherein the Irbesartan having less than about 10 parts per million (ppm) o-xylene, less than about 20 ppm toluene, less than about 20 ppm N,N-dimethylformamide, less than about 20 ppm ethyl acetate, less than about 20 ppm methyl tert-butyl ether, and less than about 10 ppm triethylamine.

20. The compound of claim 18, wherein the Irbesartan having a purity of greater than about 99.95% as measured by HPLC.

21. The compound of claim 18, wherein the Irbesartan has the overall level of organic volatile impurities less than about 200 ppm.

22. The compound of claim 21, wherein the Irbesartan has the overall level of organic volatile impurities less than about 20 ppm.

23. The compound of claim 18 which contains o-xylene or toluene at below the stated levels.

24. A substantially pure Irbesartan having tin content less than about 5 ppm.

25. The compound of claim 24, wherein the Irbesartan having tin content less than about 2 ppm.
